(19) United States
(12) Plant Patent Application Publication
(10) Pub. No.: US 2003/0208822 P1
(43) Pub. Date: Nov. 6, 2003

(54) CHRYSANTHEMUM PLANT NAMED 'GEDI TWO CAM'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/138,116

(22) Filed: May 1, 2002

(51) Int. Cl. A01H 5/00

(52) U.S. Cl. PLT/293

(57) ABSTRACT

A new and distinct Chrysanthemum plant cultivar is disclosed, characterized by a decorative-type inflorescence, consistent flowering response to short days, blooming consistently after 42 days of short day length, free branching habit, red ray florets, very uniform round growth habit, and a large quantity of blooms per flowering branch.

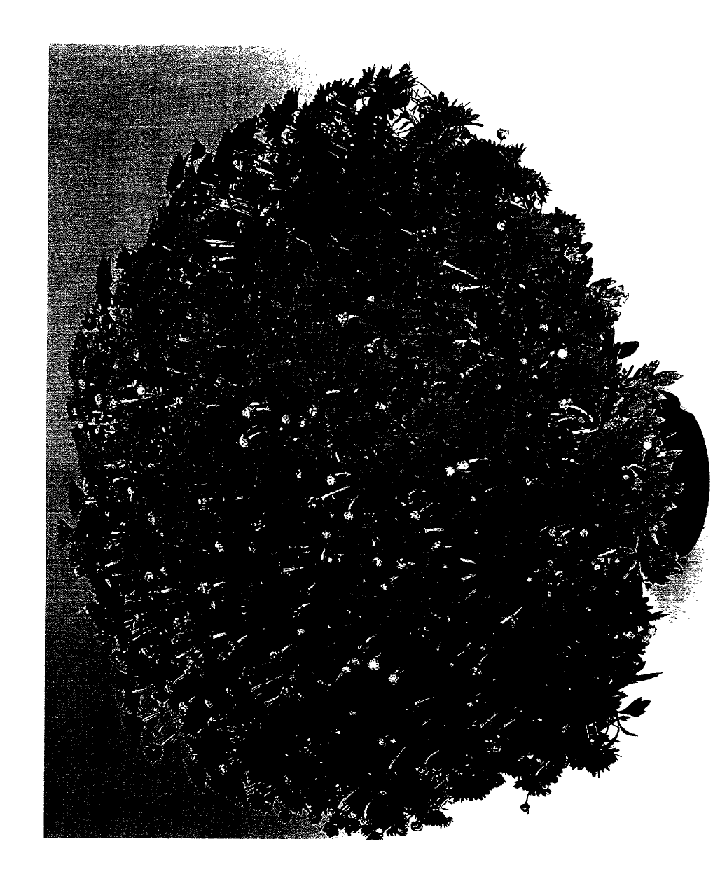

### LATIN NAME OF THE GENUS AND SPECIES

The present invention relates to a new and distinct cultivar of Chrysanthemum plant, botanically known as Dendranthema morifolium.

### VARIETY DENOMINATION

The new and distinct Chrysanthemum plant is hereinafter referred to by the cultivar name ‘GEDI TWO CAM.’

### BACKGROUND OF THE INVENTION

The new cultivar is a product of hybridization of a female parent, cultivar ‘Preco’ (undistributed in the United States), and a male parent, ‘Red Urano’ (U.S. Plant patent application Ser. No. 09/550,156). This cultivar was discovered and selected by the inventor in October 1997.

Asexual reproduction of the new cultivar by apical tip cutting and meristem tissue culture was performed in Oxnard, Calif. and has shown that the unique features of this cultivar are stable and reproduced true to type on successive generations.

### SUMMARY OF THE INVENTION

The cultivar ‘GEDI TWO CAM’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI TWO CAM.’ These characteristics in combination distinguish ‘GEDI TWO CAM’ as a new and distinct Chrysanthemum cultivar:

1. Decorative-type inflorescence,

2. Consistent flowering response to short days, blooming consistently after 42 days of short day length,

3. Free branching habit,

4. Red ray florets,

5. Very uniform round growth habit, and

6. Large quantity of blooms per flowering branch.

Plants of the new cultivar are similar to plants of the parent cultivar, ‘Preco’ in most horticultural characteristics, however plants of the new cultivar differed from plants of the cultivar ‘Preco’ primarily in ray floret color. The new cultivar ‘GEDI TWO CAM’ induces and develops blooms earlier than the male parent cultivar ‘Red Urano’ and has fewer ray florets per inflorescence than the male parent. The new cultivar also has an inflorescence which remains decorative.

In comparison to the commercially available variety ‘Jenny Wren’ (U.S. Plant Pat. No. 10,213), ‘GEDI TWO CAM’ naturally blooms one week earlier and has fewer ray florets. The new cultivar has a deep red inflorescence and more blooms per flowering branch than ‘Jenny Wren.’

### BRIEF DESCRIPTION OF THE PHOTOGRAPH

The photograph shows a plant of ‘GEDI TWO CAM’ grown in a six-inch container. One cutting was used in the pot.

### DETAILED BOTANICAL DESCRIPTION

In the following description, color references are made to The Royal Horticultural Society Colour Chart except where general terms of ordinary dictionary significance are used. The following observations and measurements describe plants grown in Oxnard, Calif. during the month of December and January. The growing temperature ranged from 55° to 68° F. at night to 60° to 75° F. during the day. Measurements and numerical values represent averages of typical flowering types.

Botanical classification: Dendranthema morifolium cultivar ‘GEDI TWO CAM’.

Commercial classification: Garden-type Chrysanthemum.

Parentage: Hybridization of a female parent, cultivar ‘Preco’ (undistributed in the United States), and a male parent, ‘Red Urano’ (U.S. Plant patent application Ser. No. 09/550,156).

### PROPAGATION

Time to rooting: 7 to 14 days at approximately 21° C.

Rooting habit: Fine, fibrous.

### PLANT

Growth habit: Mounding herbaceous perennial.

Height.—Approximately 20 cm.

Spread.—Approximately 26 cm.

Growth rate.—Moderate.

Branching characteristics.—Free Branching.

Length of lateral branches.—Approximately 19 cm.

Number of leaves per lateral branch.—Approximately 52.

### FOLIAGE

Leaf:

Arrangement.—Alternate.

Average length.—Approximately 6.0 cm.

Average width.—Approximately 4.2 cm.

Shape of blade.—Ovate.

Apex.—Cuspidate.

Base.—Attenuate.

Attachment.—Stalked.

Margin.—Palmately lobed.

Texture of top surface.—Lightly pubescent.

Texture of bottom surface.—Lightly pubescent.

Color.—Young foliage upper side: Near 137A. Young foliage underside: Near 138A. Mature foliage upper side: Near 139A. Mature foliage under side: Near 138A. Venation upper side: Near 138B. Venation under side: Near 138B.

Type.—Net.

Petiole:

Average length.—Approximately 2.5 cm.

Color.—Near 139A.

Diameter.—Approximately 0.3 cm.

### BLOOM

Inflorescence:

Flowering habit.—Induced by darkness period greater than 13.5 hours, approximately 42 days of appropriate day length required to induce and develop blooms.

Inflorescence form.—Decorative.

Natural flowering season.—Approximately from the last week of August through the first week of September.

Number of inflorescences per lateral branch.—Approximately 13.

Inflorescence diameter.—Approximately 4.8 cm.

Inflorescence depth.—1.5 cm.

Inflorescence longevity on plant.—Approximately 25 days.

Persistence.—Persistent.

Ray florets:

Appearance.—Matte.

Texture.—Smooth.

Average number per flower.—135.

Shape.—Oblong.

Aspect.—Flat.

Margin.—Entire.

Apex.—Retuse.

Length.—Approximately 2.4 cm.

Width.—Approximately 0.6 cm.

Color.—Upper surface at first opening: Near 46A. Upper surface at maturity: Near 46A. Upper surface at fading: Near 181A. Under surface at first opening: Near 180A. Under surface at maturity: Near 182B. Under surface at fading: Near 182C.

Disc florets:

Appearance.—Shiny.

Texture.—Smooth.

Average number per flower.—1.

Shape.—Cylindric.

Apex.—Obtuse.

Average length.—Approximately 0.3 cm.

Average width.—Approximately 0.1 cm.

Color.—At first opening: Near 13B. At maturity: Near 21B. At fading: Near 21B.

Peduncle:

Length.—At terminal end (shortest): Approximately 3 cm. At lateral end (longest): Approximately 5.2 cm.

Angle to stem.—Acute.

Strength.—Moderate.

Color.—Near 138A.

Habit.—Upright.

Diameter.—Approximately 0.2 cm.

Inflorescence bud:

Length.—Approximately 0.8 cm.

Diameter.—Approximately 1.0 cm.

Form.—Globular.

Color.—Near 138A.

Involucral bracts (Phyllaries):

Appearance.—Matte.

Texture.—Lightly pubescent.

Number.—Approximately 115 arranged throughout the entire inflorescence.

Shape.—Oblanceolate.

Margin.—Entire.

Apex.—Acute.

Length.—Approximately 0.8 cm.

Width.—Approximately 0.2 cm.

Color.—Upper side: Near 141B. Underside: Near 141B.

### REPRODUCTIVE ORGANS

Ray florets:

Pistillate.—Number of pistils per flower: 1. Stigma shape: 2 branched. Stigma color: Near 5A. Style color: Near 154D. Style length: Approximately 0.2 cm.

Disc florets (hermaphroditic):

Pistillate.—Number of pistils per flower: 5. Stigma shape: Two-branched. Stigma color: Near 21B. Style length: Approximately 0.3 cm. Style color: Near 154D.

Staminate.—Number of stamens per flower: 1. Anther shape: Tubular. Anther color: Near 13A.

What is claimed is:

1. A new and distinct cultivar of Chrysanthemum plant named ‘GEDI TWO CAM’ as herein illustrated and described.
